(19) United States
(12) Patent Application Publication
(10) Pub. No.: US 2020/0299744 A1
(43) Pub. Date: Sep. 24, 2020

(54) MODIFIED PROTEIN AND METHOD FOR USING SAME

(71) Applicant: HITACHI, LTD., Tokyo (JP)

(72) Inventors: Masashi MARUYAMA, Tokyo (JP); Keisuke SHIBUYA, Tokyo (JP)

(21) Appl. No.: 16/765,658

(22) PCT Filed: Jan. 30, 2019
(86) PCT No.: PCT/JP2019/003123

§ 371 Date: May 20, 2020

(30) Foreign Application Priority Data
Feb. 19, 2018 (JP) 2018-026792

(51) Int. Cl. C12P 21/00; C07K 14/62; C08F 8/30; C08F 20/56

(52) U.S. Cl. CPC C12P 21/00; C08F 20/56; C08F 8/30; C07K 14/62

(57) ABSTRACT

The present invention pertains to a means and method for, from a mixture (cell culture) of cells, a medium component, and a useful substance, recovering the first two using a single process. Specifically, the present invention pertains to a polypeptide modified with a stimulus-responsive polymer, characterized in that the stimulus-responsive polymer responds to a stimulus to induce a change in the physical properties of the modified polypeptide overall, and the change is reversible or pseudo-reversible.

### TECHNICAL FIELD

The present invention relates to a medium component that can be easily reused in cell culture. More specifically, the present invention relates to a modified polypeptide being capable of using as a medium component, and a method for using the same.

### BACKGROUND ART

In general, various cell culturing methods have been known for producing and recovering a useful substance by culturing cells such as animal cells, plant cells and microbial cells in a predetermined medium. Among them, the continuous culturing method has an advantage that, unlike the batch culturing method or the fed-batch culturing method, the step of recovering a useful substance can be continuously performed in parallel with the culturing step. In such a continuous culturing method, the productivity of useful substance can be improved by setting the cell density per unit volume of medium to be higher.

Conventionally, in order to maintain high cell density in a culture vessel in the continuous culturing method, a technique is known to return cells contained in a liquid medium withdrawn together with a useful substance from a culture vessel to the culture vessel (PTL 1). However, in the conventional continuous culturing method, it is necessary to newly add a liquid medium to the culture vessel depending on the amount of the withdrawn liquid medium.

On the other hand, a technology has been developed to reduce the amount of medium for use by removing cells from a culture medium from which a useful substance has been recovered to obtain a purified waste liquid, removing a low-molecular-weight fraction containing waste products and the like from the purified waste liquid, and returning to a culture vessel a high-molecular-weight fraction (PTL 2). However, there is a problem that the process becomes complicated because a fractioning treatment is further performed after column purification for a useful substance.

### CITATION LIST

### Patent literature

PTL 1: JP 2016-054686 A

PTL 2: JP 2010-051173 A

### SUMMARY OF THE INVENTION

### Technical Problem

Accordingly, an object of the present invention is to provide a means and a method for recovering from a mixture (cell culture product) of cells, a medium component and a useful substance the former two using a single process.

### Solution to Problem

As a result of intensive studies conducted by the present inventors to solve the above problems, it has been found that the above problems can be solved by introducing a stimulus-responsive polymer to a medium component intended to be recovered from a liquid medium withdrawn from a culture vessel and then returned to the culture vessel. Specifically, the medium component modified with the stimulus-responsive polymer will behave in the same manner as the unmodified medium component under a culture condition, while exhibiting the characteristic physical property derived from the stimulus-responsive polymer under a recovery condition to which a stimulus is applied. From a state in which cells, the medium component in a stimulus-applied state, and a useful substance are mixed, by using a separation mechanism capable of recovering the cells and the medium component in a stimulus-responding state, the useful substance can be obtained in a simple one-step process, and the cells and the media component can be returned to a culture vessel.

Accordingly, the present invention may include:

(1) A modified polypeptide with a stimulus-responsive polymer,

wherein the stimulus-responsive polymer responds to a stimulus to induce a change in the physical property of the modified polypeptide as a whole, and

the change is reversible or pseudo-reversible.

(2) A method for producing a useful substance in a cell, including:

a step of culturing a cell producing a useful substance in a culture medium containing the modified polypeptide with a stimulus-responsive polymer of (1) in the presence or absence of the stimulus; and

a step of treating a cell culture product using a semi-permeable membrane in the absence or presence of the stimulus to separate the cell and the modified polypeptide from the useful substance.

(3) A cell culture reagent, containing the modified polypeptide with a stimulus-responsive polymer according to (1).

(4) A cell culturing method, including a step of culturing a cell using a culture medium containing the modified polypeptide with a stimulus-responsive polymer of (1) in the presence or absence of the stimulus.

(5) A method for treating a cell culture product, including a step of treating a cell culture product containing the modified polypeptide with a stimulus-responsive polymer of (1), a cell and at least one secreted product from the cell using a semi-permeable membrane.

(6) A cell culture device, including:

a culture vessel into which a culture medium containing the modified polypeptide with a stimulus-responsive polymer of (1) and cells is introduced,

a separation device equipped with a semi-permeable membrane, and

a device for applying the stimulus.

### Advantageous Effects of Invention

According to the present invention, a medium component used for cell culture can be easily recovered and reused. Accordingly, the production cost of a useful substance obtained by cell culture can be reduced, and the production process can be simplified. Therefore, the present invention is useful in fields of cell culture, production of useful substances utilizing cells, production of pharmaceutical products, and the like.

### BRIEF DESCRIPTION OF DRAWINGS

FIG. 1 is a schematic view illustrating separation of a medium component using a semi-permeable membrane.

FIG. 2 is a schematic view illustrating separation of a medium component modified with a polymer using a semi-permeable membrane.

FIG. 3 is a schematic view illustrating a change in a medium component modified with a stimulus-responsive polymer under a culture condition and a recovery condition.

FIG. 4 is a schematic view showing a configuration example of a continuous culture device.

FIG. 5 is a graph showing the results of a study on the pore size of a semi-permeable membrane and the permeability of insulin modified with a temperature-responsive polymer and a useful substance (IgG).

FIG. 6 is a graph showing a comparison of the effects of insulin and insulin modified with a stimulus-responsive polymer on cell growth.

### DESCRIPTION OF EMBODIMENTS

Hereinafter, embodiments of the present invention is described with reference to drawings and the like. The following description represents specific examples of the content of the present invention. The present invention is not limited to the description, but various alternations and modifications can be performed by a person skilled in the art within the scope of the technical concept disclosed in this specification.

According to the present invention, a medium component (polypeptide) is modified with a stimulus-responsive polymer in order to easily and inexpensively recover the medium component to be added to a medium during cell culture. As a result, in the presence or absence of stimulus, the physical property (for example, apparent molecular weight) of the medium component as a whole may be reversibly or pseudo-reversibly changed. Accordingly, altering the application of stimulus in a culture condition and recovery condition allows the medium component to be easily and inexpensively recovered depending on the changed physical property.

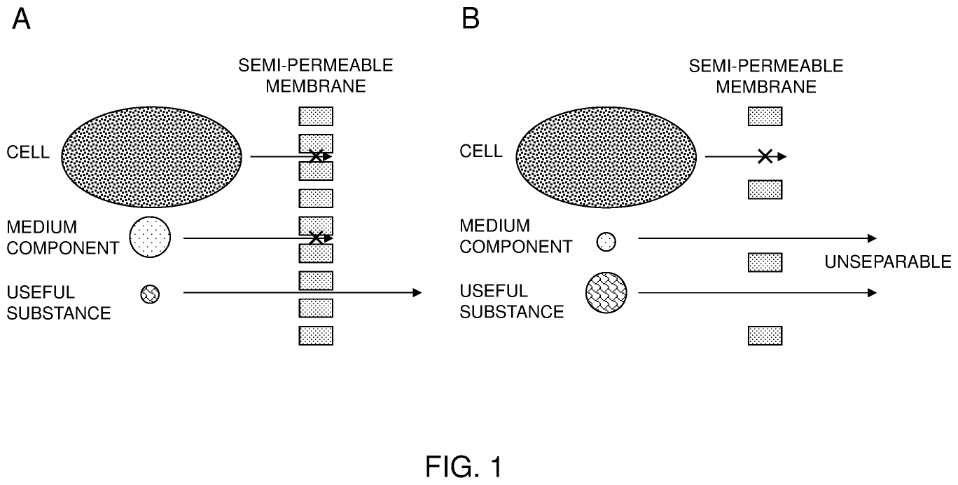

When cells are cultured to produce a useful substance, a medium component (polypeptide) is added to a liquid medium. The cells may generally be in the form of particles having a size of about several μm and in a dispersed state in a liquid medium withdrawn from a culture vessel. In the liquid medium, the useful substance and also the medium component may be in a dissolved state. Separation of a component in a dispersed state and a component in a dissolved state can be easily achieved by using a technique such as filtration, whereas separation of a plurality of components in a dissolved state may be achieved by using a difference in molecular weight or physical property. When the molecular weight of a useful substance is sufficiently smaller than that of a medium component (for example, when a useful substance is a low-molecular peptide or a secondary metabolite such as steroid), only the useful substance can be easily separated by dialysis using a semi-permeable membrane having a molecular weight cutoff between the useful substance and the medium component (FIG. 1A). However, if not (i.e., as shown in FIG. 1B, when a useful substance is larger than a medium component), such a simple method cannot be applied.

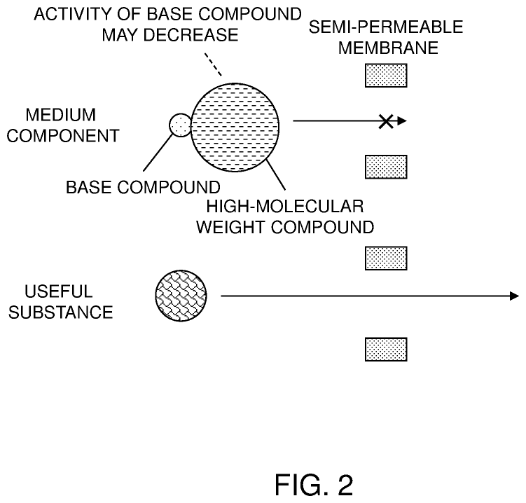

When the molecular weight of a useful substance is larger than that of a medium component, for example, it is technically possible to modify the medium component with a low-cytotoxic polymer such as polyethylene glycol (PEG) as the base compound to increase the molecular weight to be larger than that of the useful substance. However, it is likely that modifying with a high-molecular weight compound (polymer) may block the active site of the medium component as the base compound, change the kinetics, etc., thereby reducing the activity. Accordingly, it is not a generally applicable methodology (FIG. 2).

In contrast, the present invention provides a methodology by which the above-mentioned decrease in the activity of a medium component (base compound) can be suppressed. A feature of the present invention may be to modify the base compound with a stimulus-responsive polymer. In other words, the problems can be solved by achieving a stimulus responsiveness by which the stimulus-responsive polymer takes a state with little effect on the activity of the compound under a culture condition, while taking a state where a characteristic physical property is exhibited allowing for separation from a useful substance under a recovery condition.

Accordingly, in one aspect, a polypeptide modified with a stimulus-responsive polymer is provided, characterized in that

the stimulus-responsive polymer responses to a stimulus to induce a change in the physical property of the modified polypeptide as a whole, and

the change is reversible or pseudo-reversible.

The medium component (polypeptide) to be modified may not be particularly limited as long as it is a component desired to be recovered by utilizing a property that changes depending on the presence or absence of stimulus, but may preferably be a component to be added to a cell culture medium. Specifically, a component that exhibits its activity even when being modified with a stimulus-responsive polymer may be used as the modifying component, that is, the base compound. In the case of a component that expresses its activity upon incorporation into cells, although the cell membrane permeability or dynamics may change by modification with a stimulus-responsive polymer, it may be suitable as the base compound when the effect is enough small.

It may be preferable to use a high-molecular weight compound as the base compound. This is because the effect when a low-molecular weight compound is modified with a polymer is relatively larger than the effect when the high-molecular weight compound is modified with a polymer. The manner in which such an effect appears may not be limited because it depends on the relationship between a modifying polymer and a compound to be modified, but a high-molecular weight component in a medium may be most suitable as the base compound.

Accordingly, the base compound for use in the present invention may preferably be a peptide or a protein. Specifically, the base compound may be, for example, a factor that promotes cell proliferation or production of a useful substance by binding to a cell membrane protein to give a signal. Examples of such a factor may include, but are not limited to, growth factors such as insulin and transferrin, proliferation factors, hematopoietic factors, osteogenic factors and hemoproteins.

In addition to the above-mentioned insulin and transferrin, base compounds to which the present invention can be applied may also include, but are not limited to, an epidermal growth factors (EGFs), insulin-like growth factors (IGFs), transforming growth factors (TGFs), nerve growth factors (NGFs), brain-derived neurotrophic factors (BDNFs), vascular endothelial cell growth factors (VEGFs), granulocyte colony stimulating factors (G-CSFs), granulocyte macrophage colony stimulating factors (GM-CSFs), platelet-derived growth factors (PDGFs), erythropoietin (EPO), thrombopoietin (TPO), basic fibroblast growth factors (bFGFs), hepatocyte growth factors (HGFs), transforming growth factors (TGFs), bone morphogenetic proteins (BMPs), neurotrophin [neurotrophic factors] (BDNFs, NGFs), fibroblast growth factors (FGFs) and bovine serum albumin (BSA).

The medium component modified with a stimulus-responsive polymer provided according to the present invention (hereinafter, also referred to as modified protein) has a function necessary to recover from a mixture (cell culture product) of cells, the modified protein and a useful substance the former two in a one-step process.

In the present invention, the stimulus-responsive polymer may be a polymer capable of responding to a stimulus to induce a change in the physical property (preferably, molecular weight or apparent molecular weight), in which the change is reversible or pseudo-reversible. Modification with the stimulus-responsive polymer may induce a change in the physical property of the polypeptide modified with the stimulus-responsive polymer as a whole in the presence or absence of the stimulus.

In specific embodiments, the changing physical property may be a molecular weight or an apparent molecular weight. For example, when controlling the recovery of a medium component, the whole molecular weight or apparent molecular weight of the modified polypeptide as a whole under at least a recovery condition may be set to be larger than that of a useful substance to be separated, so that the modified polypeptide has a permeability lower than that of the useful substance. The molecular weight cutoff of the semi-permeable membrane may be determined by the apparent molecular weight (correlated with motility) of the solvated molecule, rather than the actual molecular weight. Accordingly, in the present invention, a polymer capable of responding to a stimulus to induce a change in the apparent molecular weight can be used as the stimulus-responsive polymer.

The stimulus for the stimulus-responsive polymer may not be particularly limited, as long as the application of the stimulus can be controlled upon culturing cells and recovering a culture component. Examples of such a stimulus may include, but are not particularly limited to, temperature changes in physiological conditions (for example, a temperature change in the range of 4° C. to 42° C.), pH changes, ionic concentration changes and diol concentration changes.

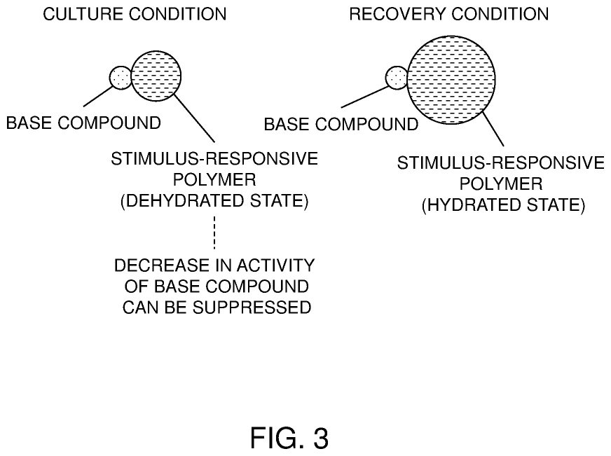

As an example, a temperature-responsive polymer can be used as the stimulus-responsive polymer. In this case, the temperature-responsive polymer may be a so-called LCST type (lower critical solution temperature type) polymer, and a responsiveness may be used in which the polymer is dehydrated and contracted at 37° C. in a culture condition, and then hydrated under a recovery condition where the liquid medium is withdrawn from a culture vessel and the temperature drops to room temperature, so that the apparent molecular weight is increased (FIG. 3). Under the culture condition, the stimulus-responsive polymer may be in a contracted state, so that there is little effect on the base compound, and under the recovery condition, the stimulus-responsive polymer may be solvated and the apparent molecular weight is increased, so that a combination can be achieved in which a useful substance passes through a semi-permeable membrane but a modified protein does not pass through the semi-permeable membrane. Since cells do not pass through the semi-permeable membrane, when such a combination can be achieved, it is possible to easily separate the cells and the modified protein (that is, medium component) from the useful substance and the like using a semi-permeable membrane.

The temperature-responsive polymers that can be used in such an application may be a polymer whose LCST is between room temperature and 37° C., and examples thereof may include, but are not limited to, polyacrylamide derivatives such as poly(N-isopropylacrylamide) (PNIPAM), polyalkylene glycol derivatives such as polyethylene glycol-polypropylene glycol copolymer, polymethacrylate derivatives such as poly(oligoethylene glycol methacrylate), polyvinyl ether derivatives such as poly(methyl vinyl ether), polyvinylamine derivatives such as poly(N-vinylcaprolactone), polyoxazoline derivatives such as poly(oxazoline), polysaccharide derivatives such as hydroxypropylcellulose, polypeptides and polypeptide derivatives. Preferably, it may be PNIPAM or a derivative thereof, which is a temperature- and pH-responsive polymer conventionally used in the art.

In addition, as another example, a pH-responsive polymer can be used. In general, during the culture, a certain amount of carbon dioxide is supplied as gas, so that a liquid medium in a culture vessel may be in a state where carbonate ions and hydrogen carbonate ions are dissolved. Accordingly, when the liquid medium is withdrawn, carbonate ions and bicarbonate ions may get out due to movement of chemical equilibrium, so that the pH of the liquid medium may change to alkaline. By using a combination in which the apparent molecular weight of the polymer increases in response to such a change, the same effect as in the above-described example using the temperature-responsive polymer can be obtained.

The pH-responsive polymer that can be used in such an application may be a polymer whose change in pH from the culture (for example, 7.2) to the recovery (for example, 7.8) affects the hydration state, and examples thereof may include a temperature-responsive polymer in which a functional group having a pKa of 2 or more and 12 or less is copolymerized, such as PNIPAM or a derivative thereof, specifically a PNIPAM-acrylic acid copolymer and PNIPAM-methacrylic acid copolymer. In such a polymer, the critical temperature of hydration-dehydration becomes dependent on the pH by controlling the molecular weight or introduction rate of a functional group having a pKa of 2 or more and 12 or less, so that a physical property in which the polymer is in a dehydrated state under a culture condition (for example, 37° C., pH 7.2), whereas the polymer is in a hydrated state under a recovery condition (for example, 25° C., pH 7.8) and the like can be obtained. When a plurality of the same functional groups are present in one molecule, the pKas may be different from each other. Even if the pKa in water when present alone does not fall within the above-mentioned range, in the case where the pKa in water when present plurally falls within the above-mentioned range, the above-mentioned pH response effect can be obtained.

As yet another example, a diol-responsive polymer can be used. In general, glucose may be added to a liquid medium as a nutrient. Since glucose is consumed during the culture, a culturing method has been developed in which the growth and activity of cells are kept high by adding glucose as appropriate. Such a culturing method can be combined with continuous culturing, and it is technically possible to separate cells and a modified protein from a useful substance by adding glucose to a liquid medium withdrawn from a culture vessel. In this case, for example, when the base compound is modified with a diol-responsive polymer having a boronic acid moiety that is a diol-responsive functional group, an increase in the molecular weight of the diol-responsive polymer due to movement of chemical equilibrium resulting from an increase in glucose concentration and an increase in the apparent molecular weight due to solvation can be expected.

In addition, as the stimulus, addition of a specific component, addition of a specific ion, light irradiation and the like are contemplated, but the stimulus may not be limited to them as long as it can induce a change in the physical property, preferably a change in the molecular weight or apparent molecular weight.

In general, when separating a protein having a different molecular weight using a semi-permeable membrane, it is considered that when the difference in molecular weight is about twice, separation is easy. In other words, it is desirable to design the medium component, the stimulus-responsive polymer used for modification, and the recovery process such that the apparent molecular weight of the medium component under a recovery condition is twice or more the apparent molecular weight of a useful substance. For example, the apparent molecular weight may be designed so as to have 300 kDa or more in a physiological condition at 25° C.

In addition, the apparent molecular weight of the medium component under a culture condition and a recovery condition can be estimated from the permeability to a semi-permeable membrane in a state simulating each condition. In other words, the permeability of a medium component and globular proteins having various molecular weights (used as standard substances) with respect to a plurality of semi-permeable membranes having different pore sizes (pore diameters) is measured. The molecular weight of a globular protein exhibiting an equivalent permeability as that of the medium component is the apparent molecular weight of the medium component under its condition. At this time, the globular proteins used as standard substances may be globular proteins generally used for measuring the molecular weight in, for example, a semi-permeable membrane or gel permeation chromatography (GPC). In addition, since it is likely that the obtained results are different depending on the material of a semi-permeable membrane to be used and the like, it may be preferable to examine such a measurement using a semi-permeable membrane actually used for separating a medium component. Although a correlation is not always obtained, it is also possible to easily measure the medium component with an aqueous GPC to estimate the apparent molecular weight using a calibration curve of the GPC for globular proteins.

On the other hand, the apparent molecular weight of a stimulus-responsive polymer under a culture condition may also be important because it affects the activity of a base compound. The decrease in the activity of the base compound due to modification with the polymer may depend on the base compound, the position for modification with the polymer, the number of modifications therewith, the molecular weight of the polymer and the like. A person skilled in the art will understand that in order to control the recovery by stimulus response while suppressing the decrease in activity, it may be desirable that the apparent molecular weight of the medium component as a whole (modified protein as a whole) change by at least about 10% or more by the stimulus response (i.e., in the presence and absence of the stimulus).

The change in physical property due to stimulus response of the stimulus-responsive polymer that can be used in the present invention may not be limited to the above-mentioned molecular weight or apparent molecular weight. An example other than the molecular weight or apparent molecular weight may include a surface charge amount. In this case, for example, a pH-responsive polymer can be used, and a responsiveness can be used in which a near neutral surface charge of the polymer under a culture condition significantly changes to a positive or negative charge under a recovery condition. In such a system, it is necessary for separation from a useful substance to use a semi-permeable membrane having a charge having the same sign as that of the charge provided by the stimulus-responsive polymer under a recovery condition, rather than a normal semi-permeable membrane. In this case, a useful substance may be separated from the medium component by using the dependence of the permeability on the charge repulsion in addition to the dependence of the permeability on the molecular size. In addition, conversely from the permeability at this time, the apparent molecular weight considering the contribution of the charge can be determined. By using the apparent molecular weight determined in this way, it is possible to set a physical property condition necessary for the effect of recovering from a mixture of cells, a medium component and a useful substance the former two, similarly to the discussion of the change in apparent molecular weight due to the temperature-responsive change in hydration-dehydration as described above. Also at this time, it is possible to estimate the apparent molecular weight by using the semi-permeable membrane as described above.

Another methodology for media component recovery can recover from a mixture of cells, a medium component and a useful substance the former two by introducing a purification tag into the base compound, filtering out the cells using a filtration membrane or the like having a component that captures the introduced purification tag, while allowing the filtration membrane to adsorb the medium component, and then eluting the medium component from the filtration membrane. For example, when, as the component for capturing the purification tag, a temperature-responsive component may be used that adsorbs the purification tag at 37° C. immediately after being withdrawn from a culture vessel and releases the purification tag at 25° C., it is possible to recover from a mixture of cells, a medium component and a useful substance the former two with a relatively simple process.

As a method for introducing a stimulus-responsive polymer to a base compound that is a medium component, a graft-from method including allowing a monomer to act on the base compound, and a graft-to method including allowing a stimulus-responsive polymer having a reactive functional group to act on the base compound can be considered. Either method may be used as long as the condition is so mild that the activity of the base compound does not decrease, but the graft-to method may be preferable from the viewpoint of reaction control and reproducibility.

For example, when a stimulus-responsive polymer is introduced into the base compound by the graft-to method, a stimulus-responsive polymer having an active ester group such as NHS ester, or a reactive functional group such as epoxy group or aldehyde group can be allowed to act on, for example, an amino group of a lysine residue. In another example, a stimulus-responsive polymer having a reactive functional group such as a maleimide group or thiol group can be allowed to act on a thiol group of a cysteine residue. In still another example, after an activating agent is allowed to act on a carboxylic acid residue to convert to an active ester, a stimulus-responsive polymer having a reactive functional group such as an amino group can be allowed to act on the active ester. Alternatively, a method such as native chemical ligation (NCL) may be used. A stimulus-responsive polymer may not be directly introduced into the base compound, but a stimulus-responsive polymer can be bound to the base compound via a linker. For example, after the base compound is biotinylated, a complex of streptavidin and a stimulus-responsive polymer may be allowed to act. Conversely, after the base compound is complexed with streptavidin, a biotinylated stimulus-responsive polymer may be introduced.

These methods for introducing a stimulus-responsive polymer, and the site for introduction and the number of introductions can be selected from those suitable for components used as the base compound.

In the present invention, since it is contemplated that the medium component into which a stimulus-responsive polymer is introduced is recovered and used again for cell culture, the media component will be exposed to repeated stimuli of culture condition-recovery condition. Accordingly, it may be preferable that the stimulus-responsive polymer is reversible or pseudo-reversible to such stimuli at least during a period in which the polymer is used for cell culture.

The polypeptide modified with a stimulus-responsive polymer of the present invention has a change in the physical property (preferably molecular weight or apparent molecular weight) in cell culture and recovery of culture component, it can be used in cell culture and production of a useful substance.

Accordingly, in another aspect, there is provided a cell culture reagent or a cell culture kit, characterized by including the above-mentioned polypeptide modified with a stimulus-responsive polymer.

In another aspect, there is provided a cell culturing method, including the step of culturing cells in the presence or absence of the above-mentioned stimulus using a culture medium containing the above-mentioned polypeptide modified with a stimulus-responsive polymer. The above-mentioned method may further include the steps of recovering the above-mentioned polypeptide modified with a stimulus-responsive polymer in the absence or presence of the above-mentioned stimulus, and culturing cells in the presence or absence of the above-mentioned stimulus using a culture medium containing the recovered modified polypeptide.

In still another embodiment, there is provided a cell culture device, characterized by including:

a culture vessel into which a culture medium containing the above-mentioned polypeptide modified with a stimulus-responsive polymer and cells is introduced,

a separation device equipped with a semi-permeable membrane, and

a device for applying the stimulus.

In the above-mentioned cell culture device,

it may be preferable that in the culture vessel, cell culture be performed under a condition where the stimulus is applied or stimulus is not applied by the device for applying the stimulus, and

in the separation device, a cell culture product from the culture vessel be processed under a condition where the stimulus is not applied or the stimulus is applied.

The cell culture reagent, cell culture kit, cell culturing method, and cell culture device of the present disclosure include, as a medium component for cell culture, the above-mentioned polypeptide modified with a stimulus-responsive polymer. Such a polypeptide exhibits, based on the nature of stimulus response, an activity suitable for cell culture to exert a favorable effect on cell growth and secretion of a useful substance by cells in cell culture, while exhibiting a different physical property from those in cell culture in response to a stimulus upon recovering cells and a useful substance, so that it can be easily separated from a useful substance and by-product (especially in one step). As a result, the polypeptide modified with a stimulus-responsive polymer which is a medium component can be recovered and reused for cell culture. Therefore, cost reduction can be achieved. In the present invention, the cell culture may preferably be a continuous culturing method, and the culture medium may preferably be a liquid medium.

In a further aspect, there is provided a method for producing a useful substance, including the steps of:

culturing cells producing a useful substance in a culture medium containing the above-mentioned polypeptide modified with a stimulus-responsive polymer in the presence or absence of the stimulus; and

treating a cell culture product using a semi-permeable membrane in the absence or presence of the stimulus to separate the cells and the modified polypeptide from the useful substance. The method may further include the steps of adding the separated cells and the modified polypeptide to the culture medium, and culturing the cells.

In yet another aspect, there is provided a method for treating a cell culture product, including the step of treating a cell culture product containing the above mentioned polypeptide modified with a stimulus-responsive polymer, cells and at least one secreted product from the cells using a semi-permeable membrane.

In the method for treating a cell culture product,

it may be preferable that the semi-permeable membrane is permeable by a secreted product from the cells and impermeable by the cells, and

the treatment is performed under a condition where the apparent molecular weight of the modified polypeptide is twice or more the apparent molecular weight of the secreted product from the cells.

The useful substance or secreted product from cells that can be produced by applying the method of the present invention may not be particularly limited as long as it is a substance conventionally produced by cell culture, and examples thereof may include immunoglobulins (IgG, IgA, IgM, IgE) and tissue plasminogen activators (tPA). The useful substance or secreted product may also be a recombinant protein or a conjugated protein.

According to the method for producing a useful substance and the method for treating a cell culture product of the present invention, a useful substance or secreted product from cells can be easily recovered from cells and a medium component (i.e., the polypeptide modified with a stimulus-responsive polymer). The separated cells and medium component can be reused, and particularly when the medium component is expensive, cost reduction can be achieved.

### EXAMPLE

In this Example, a study was conducted assuming Chinese hamster ovary (CHO) cells as the cells, a temperature-responsive growth factor (insulin modified with a temperature-responsive polymer) as the medium component, insulin as the base compound of the medium component, and IgG as the useful substance. A semi-permeable membrane was used for recovery of the former two.

### Cells and Medium

For the culture experiment, Chinese hamster ovary cells (CHO cells; CRL-9606 cells) (adherent cultured cells were acclimatized to suspension cells) were purchased from the American Type Culture Collection (ATCC) for use. The medium used was a Ham's F12 basic medium supplemented with insulin and transferrin (either at a final concentration of 10 μg/mL) and fetal bovine serum (FBS) (final concentration of 10%) (hereinafter referred to as a standard medium).

### Culture Broth Circulation Test

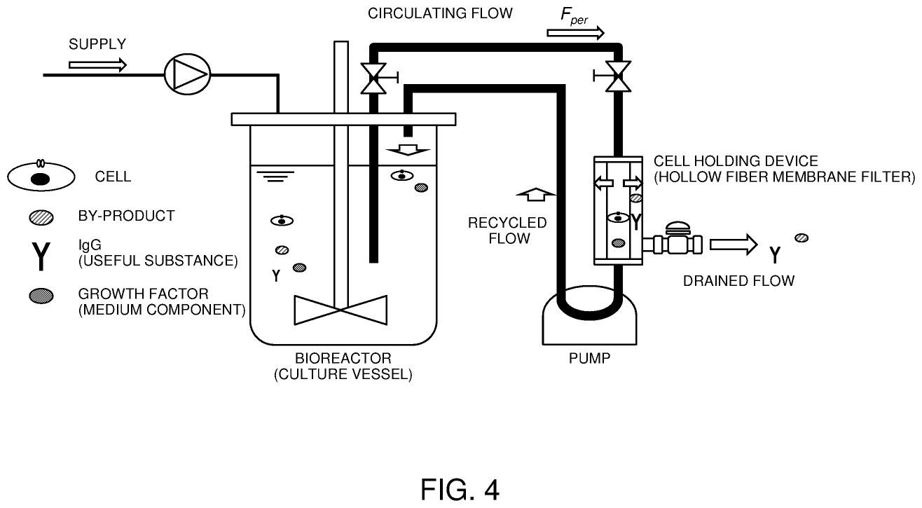

The apparatus shown in FIG. 4 was configured using a 1 L culture vessel (ABLE Corporation) and a hollow fiber filter with a pore size of 300 kDa (Spectrum Laboratories Inc.). The CHO cells were diluted to 1×10⁵ cells/mL with the standard medium, placed in the 1 L culture vessel, and maintained at a temperature of 37° C., dissolved oxygen of 2.7 mg/L and pH of 7.2, while being circulated at a predetermined circulation rate (0, 4, 10 mL/min) for cell culture for 8 days. The culture broth was sampled from the culture vessel once a day, and the antibody (IgG) concentration, number of viable cells, viability, lactic acid concentration, ammonia concentration, glutamine concentration and glucose concentration were measured.

### Preparation of Temperature-Responsive Growth Factor

Into a solution of growth factor insulin (10 mg/mL, 1.0 mL), a solution of poly(N-isopropylacrylamide)-N-hydroxysuccinimide (PNIPAM-NHS) (Mn=9.0×10³, Mw/Mn=1.8), the structure of which is changed in response to temperature, in dimethylformamide (DMF) (12.5 mg/mL, 2.5 mL, 2.0 eq.) was added. The resulting white suspension was reacted at 37° C. for 12 hours to introduce PNIPAM to a lysine residue of insulin. Hereinafter, the insulin modified with the temperature-responsive polymer PNIPAM is referred to as a temperature-responsive growth factor.

### Filter Permeability Test of Temperature-Responsive Growth Factor

The apparatus shown in FIG. 4 was configured using a 100 mL glass container (in place of the culture vessel) and a hollow fiber filter (any of three types having a pore size of 10 kDa, 100 kDa and 300 kDa) (Spectrum Laboratories Inc.). In the 100 mL glass container, a solution of the temperature-responsive growth factor diluted with a buffer solution (PBS) (concentration of 10 μg/mL) was placed and circulated at a rate of 10 mL/min using a peristaltic pump. Subsequently, the solution was withdrawn at 1 mL/min through a withdrawing outlet of the hollow fiber filter. During this time, the glass container, piping tube and hollow fiber filter were all placed in a thermostatic chamber and maintained at a constant temperature (any of 25° C., 37° C. and 42° C.). Each solution was sampled from the 100 mL glass container and through the withdrawing outlet of the hollow fiber filter, the concentration of the growth factor was quantified by an ELISA method, and the permeability was determined by the following equation (1). As a control experiment, the same test as mentioned above was performed for insulin and antibody, and the permeability was determined.

$$[ Equation 1 ]$$

$$PERMEABILITY ( \% ) = \frac{\begin{matrix}CONCENTRATION AFTER PASSING THROUGH FILTER \\ ( CONCENTRATION OF GROWTH FACTOR AT OUTLET \\ OF HOLLOW FIBER )\end{matrix}}{\begin{matrix}CONCENTRATION BEFORE PASSING THROUGH FILTER \\ ( CONCENTRATION OF GROWTH FACTOR IN 100 mL \\ GLASS CONTAINER )\end{matrix}}$$

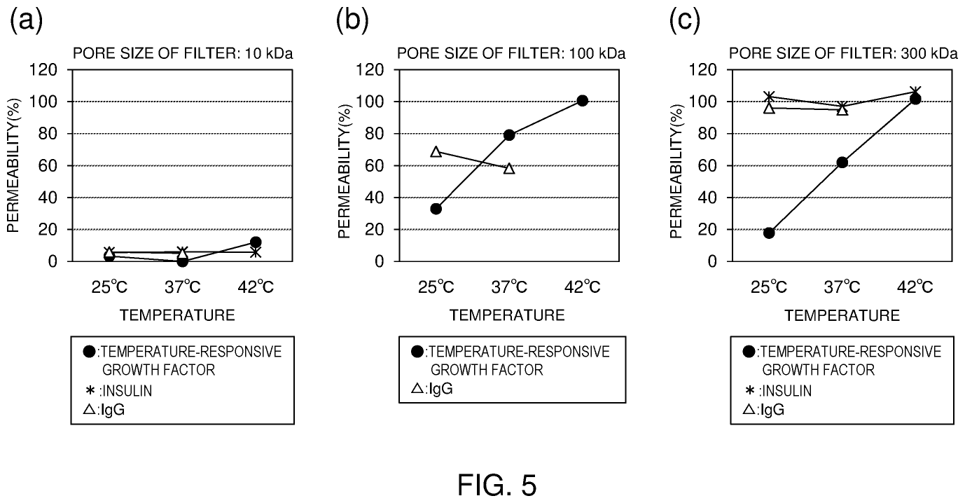

The results are shown in FIG. 5. In the case of the hollow fiber membrane having a pore size of 10 kDa, all molecules cannot pass through the membrane regardless of the temperature (FIG. 5(a)). Insulin is smaller than the pore size of 10 kDa, but could not pass through the membrane. This is presumably because the steric size of the molecule is larger. In the case of the hollow fiber membrane having a pore size of 100 kDa, the temperature-responsive growth factor has a temperature dependency of permeability, and the permeability decreases as the temperature decreases (FIG. 5(b)). This is presumably because the temperature-responsive polymer portion of the temperature-responsive growth factor transitioned to a hydrated state due to decrease in temperature, and became difficult to pass through the pores due to increase in apparent molecular weight. On the other hand, the temperature dependency of IgG was small, and the permeability was about 60% (FIG. 5(b)). This indicates that the pore size and the molecular size of IgG are relatively close to each other, and part of IgG passes through the membrane. In the case of the hollow fiber membrane having a pore size of 300 kDa, the permeability of the temperature-responsive growth factor was temperature-dependent, being 20% at 25° C. and 100% at 42° C. (FIG. 5(c)). On the other hand, since IgG and insulin have molecular sizes smaller than the pore size of 300 kDa, it can be seen that all of them pass through the membrane (FIG. 5(c)).

From the above, under these conditions, it can be seen that using the hollow fiber membrane having a pore size of 300 kDa at 25° C. upon separating cells can prevent the temperature-responsive growth factor from passing through the membrane and allow pharmaceutical product IgG to pass through the membrane.

From this result, it has been suggested that the temperature-responsive growth factor studied this time has an apparent molecular weight of about 100 kDa under a culture condition at 37° C., and has an apparent molecular weight of about 300 kDa or more under a recovery condition at 25° C. This result is reasonable since it is common for a polymer-modified protein to exhibit an apparent molecular weight much higher than the actual molecular weight.

### Flask Culture Test Using Temperature-Responsive Growth Factor

The CHO cells were seeded in a medium containing the temperature-responsive growth factor (10 μg/mL) instead of insulin in a 6-well culture plate, and cultured in an incubator (5% CO₂, 37° C.) for 5 days. The seeding density and the number of cells 5 days after the culture were measured, and the growth rate was calculated. As a control experiment, the CHO cells were similarly cultured using a medium containing normal insulin or a medium containing no insulin.

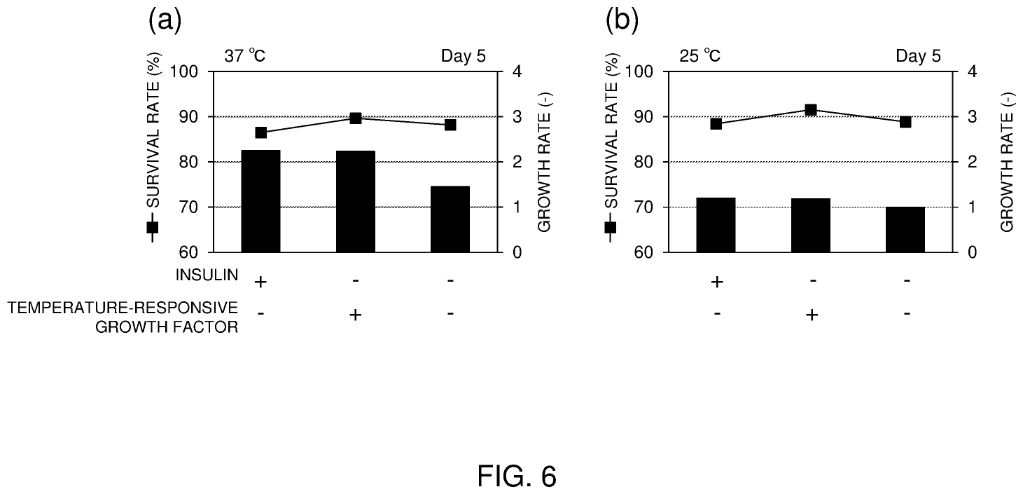

The results are shown in FIG. 6. When cultured in a medium containing the temperature-responsive growth factor at a temperature of 37° C., the growth rate was equivalent to that of the medium supplemented with insulin. This is about twice that of the culture medium without supplementation with insulin or the temperature-responsive growth factor (FIG. 6(a)). On the other hand, when cultured at a temperature of 25° C., the CHO cells did not grow in any of the medium containing the temperature-responsive growth factor or the medium containing insulin (FIG. 6(b)). The survival rate is maintained at about 90% in either culture at 37° C. or 25° C., so it can be seen that the temperature-responsive growth factor prepared in this Example does not affect the cell viability. From these results, it has been demonstrated that the temperature-responsive growth factor studied this time does not have an adverse effect due to the temperature-responsive polymer used for modification, but exhibits an activity equivalent to that of insulin before modification.

1. A modified polypeptide with a stimulus-responsive polymer, wherein the stimulus-responsive polymer responds to a stimulus to induce a change in a physical property of the modified polypeptide as a whole, and the change is reversible or pseudo-reversible.

2. The modified polypeptide according to claim 1, wherein the change in a physical property is a change in a molecular weight or an apparent molecular weight.

3. The modified polypeptide of claim 2, wherein the apparent molecular weight of the modified polypeptide as a whole changes by 10% or more in a presence and absence of the stimulus.

4. The modified polypeptide according to claim 1, wherein the stimulus-responsive polymer responds to at least one stimulus selected from the group consisting of a temperature change, a pH change, an ionic concentration change, and a diol concentration change under a physiological condition.

5. The modified polypeptide of claim 4, wherein the stimulus comprises the temperature change in a range of 4° C. to 42° C.

6. The modified polypeptide according to claim 1, wherein the stimulus-responsive polymer is at least one polymer selected from the group consisting of a polyacrylamide derivative, a polypropylene glycol derivative, a polymethacrylate derivative, a polyvinyl ether derivative, a polyvinylamine derivative, a polyoxazoline derivative, a polysaccharide derivative, a polypeptide and a polypeptide derivative.

7. The modified polypeptide according to claim 1, wherein the stimulus-responsive polymer is poly(N-isopropylacrylamide) or a derivative thereof.

8. The modified polypeptide according to claim 1, wherein the polypeptide is at least one polypeptide selected from the group consisting of a growth factor, a proliferation factor, a hematopoietic factor, an osteogenic factor and a hemoprotein.

9. The modified polypeptide according to claim 1, wherein the polypeptide is at least one polypeptide selected from the group consisting of insulin, transferrin, an epidermal growth factor (EGF), an insulin-like growth factor (IGF), a transforming growth factor (TGF), a nerve growth factor (NGF), a brain-derived neurotrophic factor (BDNF), a vascular endothelial cell growth factor (VEGF), a granulocyte colony stimulating factor (G-CSF), a granulocyte macrophage colony stimulating factor (GM-CSF), a platelet-derived growth factor (PDGF), erythropoietin (EPO), thrombopoietin (TPO), a basic fibroblast growth factor (bFGF), a hepatocyte growth factor (HGF), a transforming growth factor (TGF), a bone morphogenetic protein (BMP), a neurotrophic factor (BDNF or NGF), a fibroblast growth factor (FGF) and bovine serum albumin (BSA).

10. The modified polypeptide according to claim 1, wherein the stimulus-responsive polymer is bound directly to an amino group in a lysine residue or a thiol group in a cysteine residue of the polypeptide, or bound to the polypeptide via a linker.

11. A method for producing a useful substance in a cell, comprising:
a step of culturing a cell producing a useful substance in a culture medium comprising the modified polypeptide with a stimulus-responsive polymer according to any one of claims 1 to 10 in the presence or absence of the stimulus; and
a step of treating a cell culture product using a semi-permeable membrane in the absence or presence of the stimulus to separate the cell and the modified polypeptide from the useful substance.

12. The method according to claim 11, further comprising steps of adding the separated cell and the modified polypeptide to the culture medium, and culturing the cell.

13. The method according to claim 11, wherein the useful substance comprises at least one selected from the group consisting of IgG, IgA, IgM and IgE.
